(19) United States
(12) Patent Application Publication
(10) Pub. No.: US 2018/0313857 A1
(43) Pub. Date: Nov. 1, 2018

(54) System and Method for the Prevention, Diagnosis and Treatment of Protein Misfolding Diseases

(71) Applicant: Kavitha Reddy, Boston, MA (US)

(72) Inventor: Kavitha Reddy, Boston, MA (US)

(21) Appl. No.: 15/961,984

(22) Filed: Apr. 25, 2018

Related U.S. Application Data
(60) Provisional application No. 62/559,775, filed on Sep. 18, 2017.
(60) Provisional application No. 62/490,560, filed on Apr. 26, 2017.

(51) Int. Cl. G01N 33/92; G01N 33/50; G01N 33/18; A61K 31/25; G01N 33/84; A01K 67/027; A61K 33/08

(52) U.S. Cl. CPC G01N 33/92; G01N 33/5076; G01N 33/5091; G01N 33/1813; A61K 31/25; A61P 25/16; A01K 67/0278; A61K 33/08; A01K 2227/105; A01K 2267/03; G01N 33/84

(57) ABSTRACT

A system and method for the prevention, diagnosis, and treatment of protein misfolding diseases includes altering the binding or interaction(s) between any of the following (a) a peptide of interest in protein-misfolding disease, such as PrP-C or PrP-Sc, and (b) a mineral (c) copper, and/or d) a lipid particle, wherein the mineral may be montmorillonite or another mineral, and the lipid particle may be a vesicle or micelle, or may form or promote the formation of non-lamellar curved-lipid shapes.

### RELATED APPLICATIONS

This application is related to and claims priority from pending U.S. provisional patent application Ser. No. 62/559,775 filed Sep. 18, 2017, 2017, entitled System and Method for the Prevention, Diagnosis and Treatment of Protein Misfolding Diseases; and pending U.S. provisional patent application Ser. No. 62/490,560 filed Apr. 26, 2017, entitled System and Method for the Prevention, Diagnosis and Treatment of Protein Misfolding Diseases; each of which is hereby incorporated by reference herein for all purposes.

### FIELD OF THE DISCLOSURE

The invention relates generally to systems and methods for the prevention, diagnosis and treatment of protein misfolding diseases, and more specifically to the identification and/or use of mineral and/or lipid particles in such preventive and diagnostic methods and treatments.

### BACKGROUND

Cells rely on properly-folded proteins for numerous essential functions. Proteins can fold incorrectly due to a variety of factors, leading to disease. Human diseases of protein misfolding include Alzheimer's disease (AD), Parkinson's disease (PD), Amyotrophic lateral sclerosis (ALS), and the transmissible spongiform encephalopathies (TSEs). In animals, protein-misfolding diseases also occur in the form of prion diseases, such as Scrapie, bovine spongiform encephalopathy (BSE), and chronic wasting disease.

Transmissible spongiform encephalopathies occur in both humans and animals after exposure to a transmissible, or infectious, agent that induces abnormal protein folding of previously normal cellular protein (prion formation). The most well known human TSE disease is variant Creutzfeldt-Jakob disease (vCJD). vCJD disease occurs most commonly after consuming meat from a cow afflicted with mad cow disease (bovine spongiform encephalopathy), with resulting conversion of normal human PrP-C protein to PrP-Sc, neurodegeneration and eventual death.

The current protein-only hypothesis of prion disease suggests that PrP-Sc by itself causes PrP-C to PrP-Sc conversion. However, synthetic PrP-Sc protein alone has had difficulty in recapitulating the disease. Thus, a “protein-only” cause may be incomplete. Extracts from affected tissue transmit the disease. There may be causative or critical factors present in the extracts besides the prion protein itself. Nucleic acid appears absent; however, this observation may not exclude the possibility of additional factors beyond the misfolded protein itself. Accordingly, additional or other significant factors or agents that promote the protein misfolding may remain to be discovered.

### SUMMARY

Montmorillonite clay (Mte), a phyllosilicate or smectite clay mineral, is a common natural component of soil found in multiple regions worldwide. Mte complexes with PrP-Sc (prion) protein in the soil, providing an environmental reservoir for TSE disease. Mte appears to increase the transmission or infectivity rate of orally-ingested or mucosally-inoculated prions in Scrapie disease and chronic wasting disease affecting sheep and deer respectively. Reasons for Mte to prion soil complexing and for Mte increasing the probability of prion infectivity have not been described in detail or demonstrated. Here, Mte or other mineral particles may play a central role in the pathogenesis of TSE. Mineral particles and/or lipid particles, such as vesicles, or lipid changes may play a role in the pathogenesis of TSE, Alzheimer's disease, Parkinson's disease, and ALS.

Other benefits and advantages of the present disclosure will be appreciated from the following detailed description.

### DETAILED DESCRIPTION

Embodiments of the invention and various alternatives are described. Those skilled in the art will recognize, given the teachings herein, that numerous alternatives and equivalents exist which do not depart from the invention. It is therefore intended that the invention not be limited by the description set forth herein or below.

One or more specific embodiments of the system and method will be described below. These described embodiments are only exemplary of the present disclosure. Additionally, in an effort to provide a concise description of these exemplary embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Further, for clarity and convenience only, and without limitation, the disclosure sets forth exemplary representations of only certain aspects of events and/or circumstances related to this disclosure. Those skilled in the art will recognize, given the teachings herein, additional such aspects, events and/or circumstances related to this disclosure, e.g., additional elements of the devices described; events occurring related to protein misfolding; etc. Such aspects related to this disclosure do not depart from the invention, and it is therefore intended that the invention not be limited by the certain aspects set forth of the events and circumstances related to this disclosure.

Mineral particles and/or lipid particles or lipid changes may play a pivotal role in the prion disease transmissible spongiform encephalopathy (TSE), and also may play a similar role in other protein-misfolding diseases.

Montmorillonite clay (Mte) and other minerals are known to bind to scrapie prion protein (PrP-Sc) in soil.

In transmissible spongiform encephalopathy, Mte or other mineral particles, upon contact with lipids, such as in the gut or at the cell plasma membrane, may interact with lipids or with lipid rafts. The minerals may catalyze formation of lipid vesicles, and may also be capable of inducing lipid raft clustering or other lipid or plasma membrane changes. Some of these lipid particles, vesicles or rafts may then contain or bind PrP-Sc, and sometimes also contain montmorillonite or the mineral catalyst.

Mte-PrP-Sc or the affected lipid vesicles/particles may also promote PrP-C to PrP-Sc conversion by providing conditions for misfolding. The conditions provided for protein misfolding may include the following: multiple unique compartments providing sizes, shapes or aqueous/lipid environments that promote varied folding conformations; presence of an anion or negative-charge (Mte); lipid membrane mimetic (e.g., associated lipid vesicle or lipid raft); lipid curvature or non-lamellar shapes; interaction with bound or contained PrP-Sc; and presence of bound copper and/or copper binding affinity.

In addition to minerals, other conditions affecting lipids may promote prion disease. For example, certain lipids, even without the presence of minerals or an anion, also assemble into curved particle shapes under appropriate conditions. Lipid shapes (lipid polymorphisms, or lipid phases) include lamellar phases, and non-lamellar (curved) phases. Non-lamellar, or curved, phases include micellar, liposomal/vesicular, tubular, and hexagonal phases. According to this disclosure, lipid types that form non-lamellar curved-particle shapes, or lipids under conditions that form or promote non-lamellar curved-particle shapes, may promote prion delivery, membrane fusion, and/or protein misfolding. The provision of such lipids or the natural presence of them during infection, or the prion itself containing these lipid types, would also allow prion disease to be transmitted and to progress.

Mte-catalyzed lipid vesicles are proto-cell-like entities, making them candidates to be part of the infectious particle in prion disease, which can behave like an infectious organism but does not appear to depend on nucleic acid. Lipid vesicles or rafts may resemble endosomes, which would be consistent with reports of prion being transported and formed along an endosome-like pathway. Additionally, the lipid particles (e.g., vesicles or rafts) could accumulate intra-cellularly, and may form tubulovesicular structures, which are hallmarks of prion disease.

Potential applications of the present disclosure include protein misfolding diseases: Transmissible spongiform encephalopathies, Alzheimer's disease, Parkinson's disease, and Amyotrophic lateral sclerosis. The disclosure provides a basis for novel interventions in the field of prion and protein-misfolding diseases. In addition, this disclosure provides that silicate-or mineral-catalyzed lipid vesicles/liposomes may be useful compartments that may be manipulated to deliver molecules or even proteins to or from cells.

The Prion Protein and Transmissible Spongiform Encephalopathy

In the transmissible prion diseases scrapie and vCJD, normally-folded prion protein (PrP-C) converts to an alternate and pathologic folding conformation, termed PrP-Sc. The mammalian prion protein (PrP; major prion protein) is a C-terminally-GPI-anchored cell-surface protein. It is widely expressed on cells, though at higher levels on neurons, neuroendocrine cells, and cells of the lympho-reticular system. The full-length form, as well as likely processed truncated forms of the protein, are expressed on the cell surface. The physiologic function of the protein is not yet fully understood. In TSE, normal host wild-type PrP-C is converted to scrapie prion protein (PrP-Sc), the protein of identical sequence but differing (likely beta sheet) folding conformation, by an unknown mechanism.

A “protein-only” hypothesis to explain how PrP-C converts to PrP-Sc has been described, but has several limitations. This hypothesis suggests that abnormally-folded PrP-Sc protein itself is the transmissible and infectious agent causing TSE disease. However, exposure to PrP-Sc alone results in only limited conversion of normal PrP-C. Further and of equal interest, it has been demonstrated that PrP-Sc can actually be formed without provision of pre-existing PrP-Sc, using a protein misfolding cyclic amplification (PMCA) technique relying on PrP-C, lipid molecules, and a synthetic polyanion such as use of poly(A) RNA substrate. Teflon beads increase the amplification rate in prion PMCA reactions (PMCAb) for unknown reasons, as does addition of the amphipathic glycoside saponin. Thus, an as yet unidentified polyanion may be a component of the infectious prion particle. The search for a more complete understanding of the causative agent(s) of PrP-C to PrP-Sc conversion continues.

In nature, infected animals deposit prions into the environment, including the soil, through deposition of urine, feces, saliva, blood and tissue, especially upon death. Prion disease is then transmitted to other susceptible animals or humans, most commonly after mucosal or dietary exposure. Montmorillonite (Mte) clay, also called Fuller's earth, bentonite clay, or smectite clay, is present naturally in certain soil areas and binds to infective prions in the soil. Other soil minerals also bind to prions. Soil clay content, including content of Mte or phyllosilicate, is correlated with prion infection probability. Mte, when lyophilized with diseased brain homogenate, increases prion infectivity (using Mte particles ranging in size from the majority less than 1 micrometer to others up to 10 micrometers). An increased transmission of prions when Mte is bound has been seen. Upon oral ingestion of prions, PrP-Sc bound to Mte is associated with greater delivery of PrP-Sc from the stomach to the intestine and greater uptake of PrP-Sc to intestinal and lymphatic cells than unbound PrP-Sc. The reasons or mechanisms behind these observations have not been determined nor any hypotheses been published.

Montmorillonite Clay, and Vesicle Growth and Division

Mte is a clay mineral. Clays and other minerals (especially silicates) catalyze the formation of protocell-like vesicles when experimentally added to fatty acid micelles. Clay also catalyzes some prebiotic or biological reactions, such as the formation of RNA from activated ribonucleotides. In the presence of fatty acid micelles, clay and RNA have been shown to become encapsulated into some of these vesicles. In some of the vesicles, RNA alone can be found. Under acidic conditions, the vesicles can grow. If the vesicles are extruded through smaller pores, smaller vesicles proliferate, many with contents remaining within them. The contents inside the vesicle may also be able to “replicate” along with vesicle growth.

Therefore, clay added to fatty acid micelles may result in assembly of vesicles that can contain a catalytically active surface within a membrane vesicle, may hold particles or molecules, and may divide and multiply in a process reminiscent of primordial cells. The possibility of a natural analog of this process in the modern world seemed remote to those who described this phenomenon, as it is not obvious how this might apply in-vivo to modern biological systems. However, a related process may have occurred during the initial phases of prion disease inception and may occur in modern-day prion disease.

In addition to clay-induced lipid vesicles having been observed, semi-permeable clay vesicles have also been described and may also be present in the soil and certain environments or compartments. Organic liquids and surfactants can facilitate clay vesicle formation. Clay vesicles may be created upon exposure of Mte to water or other liquids, sonication to form nanoparticles, and exposure to shear forces such as shearing between glass slides. They likely maintain their structural stability even when dry, as well as after multiple dehydration and rehydration cycles. The walls are 8-10 nanometers thick and the vesicles are often 30-40 nanometers in size upon hydration. When exposed to glycerol, formamide, or pure water, clay bubbles do not form vesicles. Liposomes form inside the vesicles, often do not leave and can cause evolution into complex internal structures.

Clay vesicles or clay-induced lipid vesicles, or a combination of both, may have an active role in prion disease as part of the infectious particle. Clay or other mineral-induced or altered lipid particles, micelles, rafts, or vesicles may enhance cellular uptake of clay and prion proteins, promoting prion infection. The associated lipid particles may promote prion infection by delivering large amounts of PrP-Sc to the cell surface to interact with PrP-C, causing its conversion by the interaction of PrP-C with PrP-Sc. Mte and its associated traits may create favorable conditions for protein misfolding that catalyze PrP-C to PrP-Sc conversion.

Mineral particles such as Mte, mineral-altered lipid particles, and/or other lipid particles, especially those that promote formation of a non-lamellar or curved shape, rather than PrP-Sc alone, may be unrecognized component(s) of the infectious or active particle in prion disease. The following steps may occur in some forms of TSE disease. PrP-Sc may serve as a “guide” or targeting moiety that binds PrP-C, therefore causing a greater delivery to and effect of the infectious particles on PrP-C rich cells, such as neurons. Mte, a natural anion, upon contact with lipids, may catalyze the formation of lipid vesicles, or alter, induce curvature of, or mobilize lipid particles or rafts. Other conditions may also promote the formation of lipid vesicles or non-lamellar curved lipid shapes. For example, certain lipids (e.g. lysophospholipids, phosphatidylcholine, sphingomyelin, phosphatidylserine, phosphatidylglycerol, phosphatidylinositol, phosphatidic acid, cardiolipin, phosphatidylethanolamine) without the presence of minerals or an anion also assemble into non-lamellar curved particle shapes under the appropriate conditions, and therefore may promote prion delivery or membrane fusion, and/or protein misfolding. The provision of such lipids or the natural presence of them during infection, or the prion itself containing these lipid types would also allow prion disease to be transmitted and to progress. Lipid particles may protect or stabilize PrP-Sc and improve its delivery and spread, such as through promotion of membrane fusion, endocytosis or uptake, and/or intracellular and extracellular transport. Disease may be promoted by the delivery of a sufficient “seed” amount of PrP-Sc. Mineral-altered or curved lipid particles may promote uptake of PrP-Sc by the cell. The interaction of Mte/lipids and PrP-C, likely along with PrP-Sc, and possibly copper, may convert PrP-C to PrP-Sc and may stabilize the prion form. Alternatively, lipid vesicles/particles containing or bound with PrP-Sc, some possibly without Mte, may similarly convert and/or stabilize the prion form. It is possible that some lipid particles without PrP-Sc may have the conditions to promote a misfolding of PrP-C to PrP-Sc. Mte/mineral itself, or Mte/mineral-altered lipid particles, such vesicles or rafts, or other lipid particles, especially those that promote formation of non-lamellar curved-shape lipid particles, may also serve as a nidus for concentration and/or aggregation of PrP-Sc. Clay vesicles may alternatively or also participate in some of the above steps.

In accordance with the disclosure, Mte and its effects on lipids, or the presence or induction of other lipid particles, especially non-lamellar curved-shape lipid particles, may participate in the active propagation of prion disease (PrP-C to PrP-Sc conversion). This has not been previously described. Lipid particles, which may be dynamic or vary in size and shape, may provide multiple environments in which PrP-C or PrP-Sc can change shape and exhibit multiple folding conformations. Ultimately, some of these varied lipid environments may be able to convert host PrP-C more effectively and/or may produce more pathogenic folding conformations, or cause different pathologic and clinical effects, thus explaining strain variations, and how species barriers are overcome as the “pathogenic” folding conformation for that particular host may eventually develop and spread with sufficient exposures and incubation time. Another explanation for strain variations that may be considered is that the type of mineral or the type of cofactor acting as a disease catalyst influences the associated disease outcome.

One could consider that while protein-only is sufficient to convert PrP-C to PrP-Sc, this would be anticipated to occur through somewhat random, limited contact of the inoculated PrP-Sc with host PrP-C molecules. In such a protein-only scenario, PrP-Sc and disease would increase proportionally with the amount of PrP-Sc provided, and with incubation time provided for PrP-Sc to reach multiple PrP-C molecules. If cofactors are supplied, these would make clinical disease more likely to develop (increase infectivity) and decrease the time to onset, because they would stabilize the PrP-Sc conformation and likely also help distribute PrP-Sc much more widely.

This may explain, for example, how certain PrP-C mutations that make the protein prone to misfolding may produce “inherited” CJD through spontaneous misfolding of a few initial PrP-C proteins that can slowly progress to disease. As PrP-Sc accumulates, more PrP-Sc (and possibly associated lipid particles) are present and of sufficient amount to “seed” more rapidly another host upon inoculation, and the disease appears “transmissible”. If one tried to inoculate across species, if the PrP-Sc conformations and sequences were sufficiently similar that they readily interact to cause misfolding of host protein, disease would appear. Even if clinical disease does not appear in any or all cases, it is possible that with continued incubation time a few small Prp-Sc molecules can eventually change conformation to become capable of interacting with host PrP-C, possibly aided by lipid particle dynamics that permit an “evolution” of the protein conformation. If mineral such as Mte is present, this further aids in the process of accelerating lipid distribution and PrP-Sc spread as well as opportunity for new conformations to appear.

This mineral-prion particle may have an ability to behave as part of the primordial-cell-like entity that can persist in soil, propagate PrP-Sc in a host animal or human, possibly using host lipids, and then be transmitted again via ingestion or inoculation of affected tissue or return to the soil, only to begin the infective cycle anew. The physical properties of this particle may convert PrP-C to PrP-Sc and/or allow delivery of significant amounts of PrP-Sc that permit further PrP-Sc propagation, resulting ultimately in progressive cellular changes that appear to us to be similar to those of an infectious organism.

Mte and other minerals are bound to PrP-Sc naturally in soil. Epidemiologic studies show an association of soil clay content with prion disease rates. Mte increases infectivity rates of inoculated prions in animal models. Mte and other minerals (e.g. silicates) are capable of catalyzing protocell-like lipid vesicles capable of division and growth. Mte has a high affinity for and adsorbs copper. PrP protein is likely a copper-binding protein, and copper binding can promote misfolding. Lipid membrane mimetics promote PrP misfolding. Mte is an anion and can form a negatively charged surface, as can other similar minerals. An anion and lipids are able to convert PrP-C to PrP-Sc in PMCA reactions. Analysis of the infectious particle suggests it is not separable from lipid and has a negative charge.

Negatively Charged Surfaces and Lipids

The presence of negatively charged beads aids in catalyzing the conversion of PrP-C to PrP-Sc in-vitro. Clay vesicles or clay-containing lipid vesicles likely also have negatively charged surfaces. PrP may have a strong affinity for negatively charged surfaces such as mineral clays. In this disclosure, mineral particles also may serve as a negatively-charged catalyst for PrP-C to PrP-Sc conversion. Mte particles have a large surface area, a negative charge, and can be found in an appropriate size (e.g. less than 1 to 10 um) to potentially catalyze conversion.

As discussed above, Mte and other silicate minerals are able to catalyze the formation of lipid micelles and vesicles which resemble primitive cells or proto-cells having a lipid membrane. PrP-C has been shown to bind to acidic lipid-containing membrane vesicles under experimental conditions with pH-dependence, producing an ordered change in the N-terminal domain, along with a destabilization of the C-terminal domain. Binding of clay-catalyzed lipid vesicles to PrP-C may promote conversion to PrP-Sc. Alternatively, it is possible that mineral particles may alter the lipid membrane or lipid rafts, leading to changes in PrP conformation and conversion to PrP-Sc. Lipid rafts, sometimes also called detergent-resistant membrane fractions (DRMs), are specialized lipid clusters in which PrP-C and other GPI-anchored proteins often reside. The clusters are often enriched in sphingolipids and cholesterol, and appear to float freely within the fluid lipid bilayer. Lipids or lipid rafts may be necessary for PrP-C to PrP-Sc conversion. For example, PMCA reaction using lipids, synthetic polyanion, and PrP-C has been shown to produce PrP-Sc.

Role of Copper in Misfolding

Copper readily adsorbs to montmorillonite and other mineral silicates. Mineral exposure to copper may be present in the environment, and increased through copper mining, use of copper solutions such as copper sulfate or ammonia solutions in manure/fertilization, or through exposure of water to pollution with heavy metal ions. Cu-Mte and Ca-Mte are able to exert strong catalytic effects. For example, they produce glycine oligomerization and glycine-alanine hetero-oligomerization. PrP-C normally binds copper, and copper is believed to have a function in its normal physiologic role. Copper is needed for PrP-C to be endocytosed from the extracellular space into the cell. Copper, pH, and lipid/water interfaces are believed to play critical roles in prion pathogenesis, though in what manner has been unclear.

PrP protein has been found to contain two binding sites that have a high affinity for copper: one in the N-terminal octa-peptide repeat segment, and a second copper-binding site around histidines 96 and 111, which is a region crucial for prion propagation. Copper binding to the histidine- and glycine-rich octarepeat N-terminal domain of PrP produces oxidation and aggregation. Copper binding to the second site of the molecule around histidines 96 and 111 is considered important for prion formation. Copper oxidation of histidine can convert it to aspartate. Metal-catalyzed oxidation of PrP leads to a change in conformation and to aggregation; metal catalyzed oxidation could potentially convert PrP-C to PrP-Sc. PrP binding to DPC micelles, in the presence of copper, produces a conformation change of identical well-defined loops linked by Gly-Gly-Gly sequences. Residues 23-28 and 156-169 are also considered important for folding into PrP-Sc. Tris and phosphate can be used to compete with peptide for copper binding. Glycine plays an important role in PrP misfolding, and glycine binds strongly to copper.

This disclosure describes the possibility that PrP-C or PrP-Sc binds to Mte, or to mineral-catalyzed lipid vesicles or altered- particles such as lipid rafts. Copper on either PrP or adsorbed to Mte could aid in this binding. Binding might occur via the N-terminus, where glycine repeats are present and glycine-copper adsorption may aid in the binding. The now altered C-terminus conformation may then be susceptible to further misfolding, possibly through activity of chaperones or interaction with PrP-Sc molecules. Binding may occur at the second copper-binding site described above, which may promote prion formation. Besides being a common binding factor for both PrP and Mte, copper may also be important as it provides catalytic activity. Copper catalyzes glycine oxidation reactions, and may explain the tendency toward misfolding of the PrP protein. Bentonites or aluminosilicate clays can produce peptide bond formation in the presence of copper, polymerizing glycine and also to a lesser extent alanine. Interestingly, deer with glycine-rich PrP genotypes exhibit more widespread, rapid, and intense prion uptake (GG>GS>SS) than those with more serine-rich PrP types. The reason for the increased prion uptake observed in the glycine-rich genotype deer may be because of glycine-copper interactions.

Mineral particles, clay vesicles or mineral-induced/altered lipid particles may be present in humans or animals after ingestion of minerals or mineral-PrP-Sc complexes. The combination of water, acidic conditions, and fatty acids or other lipids may be present in the digestive tract or stomach. When mineral, especially silicate, particles are added, this mixture may result in micelle and vesicle formation, replication and division. With a large supply of water and other lipid micelles to add to their membrane, these vesicles could swell to a larger size. PrP-Sc may be protected inside or bound to vesicles; the vesicles may serve as large negatively-charged lipid bilayers, allowing Mte-complexed-PrP-Sc and/or PrP-Sc alone to stay inside or otherwise be more protected from protease degradation. Vesicles or particles having a combination of lipid, mineral, and/or prion may become squeezed, such as in transit through tight junctions or via endocytosis or through simple mechanical actions of the GI tract, and generate multiple smaller particles. In addition, mineral vesicles may have pores in the walls, which could participate in pore-pressure induced division of vesicles. Upon reaching the intestinal cells, if vesicles are indeed present, they may facilitate uptake into the cell. The vesicles may serve to allow delivery of PrP-Sc of higher titers or convert cell-surface PrP-C to PrP-Sc in high titers, providing a large “seed” or titer of PrP-Sc to the cells. If PrP-Sc itself is in fact a template for PrP-C misfolding as the protein-only hypothesis suggests, then this large seed of PrP-Sc may be more successful in starting the process of additional PrP-Sc conversion and overwhelming natural clearance mechanisms.

To maintain its misfolded state, PrP-Sc likely must be bound to a membrane or similar entity. The lipid vesicles or described lipid particles may resemble a lipid membrane, and help convert PrP-C to PrP-Sc, stabilize PrP-Sc, or both. Mte or other minerals adsorbed with copper may provide copious opportunities for copper-assisted misfolding of PrP-C, for example via oxidation or via copper binding. Catalytic effects of Mte and other silicates on the prion protein are also possible that may cause changes leading to misfolding.

Prions localize to lymphoid tissues prior to CNS infection. Gut-associated lymphatic tissue (MALT or GALT) can take up nano- and micro-particles by a variety of methods, which may establish prion infection in the Peyer's patches and then spread further subsequently. Nano-capsular particles may deliver proteins into cells. PrP-Sc uptake and spread of TSE infection is believed to be dependent on lipid-raft-dependent-macropinocytosis, involving the N-terminus.

Conversion may also or otherwise occur at the plasma membrane. PrP-Sc accumulates at each of these sites, as well as likely at the Golgi apparatus. Once intracellular, one might consider that PrP-Sc and/or PrP-Sc-associated vesicles may follow an endocytic pathway with multiple overlapping fates, such as routing back to the extracellular space or plasma membrane, delivery to the Golgi apparatus, delivery to lysosomes, or escape to the cytosol.

Mte- or lipid-PrP-Sc complexes may accumulate in negatively charged or endocytosis-rich sites such as lymphoid tissue and neurons. They may have been taken up by various possible mechanisms, including autophagy. After cellular internalization, they may not be degraded well. The trapping of liposomes and the formation of complex vesicular and micellar structures seen with clay vesicles or with clay-induced vesicles may be analogous to the process occurring in tubulovesicular structures (TVS), which are hallmarks of prion disease. Tubulovesicular structures (also called tubulovesicular bodies, or scrapie-associated particles; size range 20-50 or 100 nm; average 27-35 nm in diameter) are ultrastructural hallmarks seen on electron microscopy, unique to TSE. TVS occur early and are characteristic when found. The composition and cause is unknown. Isolation and purification had not been successful as of one report in 2008. Spherical particles 30-60 nm in diameter and smaller spherical particles 8-20 nm in diameter stained by ruthenium red have been found in scrapie-infected tissue. This disclosure suggests that the TVS structure, the composition of which has been unknown, may contain Mte or other minerals, PrP-Sc, and/or lipid.

In addition to TVS, autophagic vacuoles are often present in prion disease. Cationic liposomes and cationic polymers induce autophagy and formation of tubulovesicular autophagosomes with intact liposomes within. A similar process may occur with cell penetrating peptides (CPP). These vacuoles may be an indication of the autophagic pathway being activated in response to clay-induced lipid vesicles or clay-altered lipid particles, and may explain overlaps seen between autophagy and prion disease.

Experimental Testing

For increased understanding of the present disclosure, a number of experiments may be useful.

Experiment #1: Detect montmorillonite or mineral particles (including nanoparticulate material) in prion-infected tissue or lysate. Also, seek to detect mineral-altered/abnormal lipid rafts, micelles, vesicles, or lipid particles.

Experiment #2: Perform PMCA reaction, using lipids, PrP-C, and Mte particles, and assay for conversion of PrP-C to PrP-Sc.

Experiment #3: Add Mte-PrP-Sc complexes from soil to fatty acid, lipids, and water conditions followed by shearing, shaking, and/or sonication. Determine if vesicles or micelles are produced and assess the size, composition, and clay and PrP-Sc content.

Experiment #4: Treat infectious prion cell lysate with agent(s) that disrupt lipid vesicle/micellar conditions. Expose normal cells or tissue to the treated lysate and assess whether infectivity is reduced or absent.

Applications

Implications or applications of the present disclosure include, without limitation, the following.

1. Elucidation of prion disease pathogenesis and lifecycle.

2. Using minerals, mineral-catalyzed changes, and/or lipid vesicles or other lipid particles, especially those that can form non-lamellar curved shapes, to create in-vitro prions (e.g. PrP-Sc) or in-vivo prions, or both. For example, using minerals, mineral-catalyzed changes, and/or lipid vesicles to create animal models of protein-misfolding disease.

3. Protecting against and treating prion diseases. Examples include:
A. Treatments that compete for peptide, PrP-C, or PrP-Sc binding to minerals, copper, or lipids, or otherwise release the binding/interaction between any of these factors.
B. Treatments or devices that disrupt micelles or vesicles or that disrupt other non-lamellar curved lipid shapes or lipid particle structures/shapes.
C. Treatments that remove or decrease lipid particles, including treatments that reduce or alter lipid particle formation/growth, uptake and/or spread.
D. Methods that change pH, for example reduce or prevent an acidic environment.
E. Methods that decrease or prevent copper binding conditions. Examples include: (i) reduce exposure of soil and minerals to copper e.g. by reducing copper sulfate or ammonia manure usage; (ii) reduce metal pollution of water (iii) use of copper-free instruments in surgical procedures; (iv) use of filtration or copper-scavenging agents to treat water, soil, animal products or humans.
F. Use of filtration or other methods to remove Mte or other minerals from fields, water, animal feed, animal products, or even affected humans and animals.
G. Design of mineral-catalyzed or other lipid particles containing or delivering anti-prion treatment. For example, protein-modifying or protein-destructive agents, anti-oxidants, competitively binding peptides or ligands, or charge-altering agents.
H. Design of lipid particles or prion-like particles intended to interfere with natural prion transmission, activity, and/or uptake. For example, use of a lipid carrier and PrP of non-toxic or less-toxic conformation as an intervention to prevent or reduce contraction or progression of disease.
I. Addition of modifiers such as polyethylene glycol (PEG) or other molecules to reduce or clear the hypothesized lipid particles. Also engineering or causing modification of liposomes or lipid particles, such as modification of lipid types, lipid environment, lipid shapes, circulation time, temperature sensitivity, pH-sensitivity or other characteristics of liposomes and lipid particles.

4. Designing diagnostic and screening assays for prion diseases. For example, assay for Mte or mineral particles or the hypothesized prion-lipid particles in samples of interest, such as blood, bodily fluids, tissue, or in soil, or in meat.

Another example would be to test a given tissue, meat, or fluid sample for the capacity of some of the lipid particles to behave in a manner consistent with mineral-catalyzed lipid particles, e.g. to grow, change shapes, merge and/or divide.

Another example would be the use of Mte or minerals to bind prions that are present in a biological sample, and thereby detect them.

5. Delivery of proteins or other cargo (for example, ribonucleic acid (RNA), deoxyribonucleic acid (DNA), peptides or other molecules) to cells using mineral-catalyzed lipid vesicles/liposomes, or using minerals to alter lipids, for example to promote uptake, or using lipids that form or promote the formation of non-lamellar curved shape lipid particles, again for example to promote uptake.

Alzheimer's Disease, Parkinson's Disease, and Amyotrophic Lateral Sclerosis

Alzheimer's disease (AD) is the most common human dementia. It is a neurodegenerative disease of protein misfolding that shares several similarities to prion diseases. Both are diseases of abnormal protein folding. Both have a long duration before disease onset. Both appear to have prominent roles for copper in peptide binding, aggregation and misfolding. Parkinson's disease (PD) and Amyotrophic lateral sclerosis (ALS) also share many of these same features. TSE, AD, and PD all may involve altered lipid-related interactions that affect the relevant protein misfolding. There is accumulating significant evidence that AD, PD, ALS and vCJD may in fact all fall into the category of prion disease. Unlike TSEs, transmissibility is not clinically observed in AD, PD, or ALS. However, AD can be experimentally “transmitted” to other brains via inoculation, indicating a possible transmissible or non-fixed causative agent. The primary causative agent(s) of these diseases have not been identified and the trigger for abnormal protein folding in each remains poorly understood.

This disclosure describes that in Alzheimer's disease, Parkinson's disease, and/or Amyotrophic lateral sclerosis, aluminosilicates, or another mineral or anion, may catalyze the formation of lipid vesicles. Alternatively or adjunctively, minerals may interact with and alter lipid components of the cell, such as cholesterol micelles or lipid rafts. Other conditions may be present in these diseases that promote the formation of lipid vesicles or non-lamellar curved-shape lipid particles. For example, certain lipids without the presence of minerals or an anion also assemble into curved particle shapes under the appropriate conditions, and therefore may promote delivery of misfolded protein or membrane fusion, and/or protein misfolding. The provision of such lipids or the natural presence of them, would also promote disease. Lipid particles or vesicles may bind the key disease-related proteins such as alpha-synuclein, APP, or superoxide dismutase, or their intermediates, processed forms, or alternate forms, such as Abeta. Copper may assist in or mediate binding between minerals or lipids and the disease proteins. These lipid particles may promote formation, spread of and/or transport of abnormal proteins in the cell and between cells. Mineral, lipid and/or the associated copper binding may provide sites for alternate processing of protein, promote oligomerization and clustering of peptides, and/or may cause conversion to and/or stabilize misfolded prion protein forms or intermediates. Critical related factors (such as GM1 in AD; free or bound copper), could be recruited to these lipid particles and aid in prion formation, aggregation, and disease progression. Mineral particles or the hypothesized non-lamellar curved lipid particles may also serve as a nidus for concentration and/or aggregation of abnormal protein forms. Clay vesicles may also be present and may participate in some of the above steps. Minerals may be one of many possible agents or conditions that may affect lipid types, sizes, shapes, mobility, distribution, growth and/or division.

Experimental Testing

For increased understanding of the present disclosure, a number of experiments may be useful.

Experiment #1: To study AD, PD, and ALS diseases, observe the binding of the normally folded disease-related proteins and prions to various minerals, including silicates, with and without copper. Also observe the binding of these proteins to mineral-catalyzed lipid vesicles and to mineral-exposed lipid particles or rafts, with and without copper.

Experiment #2: Detect mineral particles (including nanoparticulate size particles) or altered lipid micelles or vesicles (differentiate from physiologic vesicles) in brain or neuronal tissue affected by AD, PD, and ALS. Differentiate these from healthy controls.

Experiment #3: Isolate these mineral or lipid particles from diseased tissue and re-introduce these particles into healthy tissue to confirm whether they can cause disease.

Experiment #4: Expose cells to minerals such as silicates and other minerals, and to fatty acid or lipid micelles, with and without added prion proteins such as Abeta, and observe for disease development.

Applications

Implications or applications of the present disclosure include, without limitation, the following.

1. Elucidation of Alzheimer's disease, Parkinson's disease, Frontotemporal dementia, and/or Amyotrophic lateral sclerosis disease pathogenesis.

2. Use of minerals, mineral-catalyzed changes, and/or lipid vesicles, or other lipid particles, especially those that can form non-lamellar curved shapes, to create in-vitro misfolded proteins (e.g. Amyloid beta (Abeta), alpha-synuclein, superoxide dismutase (SOD1)), or in-vivo misfolded proteins (e.g. Abeta, alpha-synuclein, SOD1), or both. For example, using minerals, mineral-catalyzed changes, and/or lipid vesicles to create animal models of protein-misfolding diseases (e.g. Alzheimer's disease, Parkinson's disease, Amyotrophic lateral sclerosis).

3. Protecting against and treating protein misfolding diseases. Examples include, without limitation:
A. Treatments that compete for diseased or normal peptide/protein (e.g. Amyloid precursor protein (APP), Abeta, SOD1, alpha-synuclein) binding to minerals, copper, or lipids, or otherwise release the binding/interaction between any of these factors.
B. Treatments or devices that disrupt micelles or vesicles or that disrupt other non-lamellar curved lipid shapes or lipid particle structures/shapes.
C. Treatments that remove or decrease lipid particles, including treatments that reduce or alter lipid particle formation/growth, uptake and/or spread.
D. Methods that change pH, for example reduce or prevent an acidic environment.
E. Methods that decrease or prevent copper binding conditions. Examples include: reduce exposure of soil and minerals to copper e.g. by reducing copper sulfate or ammonia manure usage; reduce metal or copper pollution of water; use of copper-free instruments in surgical procedures; and use of filtration or copper-scavenging agents to treat water, soil, animal products or humans.
F. Use of filtration or other methods to remove Mte or other potentially harmful minerals or mineral-forming precursors from human consumer products such as cosmetics, from additives or foods intended for human consumption, from fields/soil, water, animal feed, animal products intended for human consumption, or even from affected humans and animals.
G. Design of mineral-catalyzed or other lipid particles as disease prevention or treatment. For example, particles containing protein-modifying or protein-destructive agents, anti-oxidants, competitively binding peptides or ligands, or charge-altering agents. Design of lipid particles intended to interfere with natural disease transmission, activity, and/or uptake. For example, use of a lipid particle of non-toxic or less-toxic conformation as an intervention to prevent or reduce contraction or progression of disease.
H. Addition of modifiers such as polyethylene glycol (PEG) or other molecules to reduce or clear disease-promoting lipid particles. Also engineering or causing modification of liposomes or lipid particles, such as modification of lipid types, lipid environment, lipid shapes, circulation time, temperature sensitivity, pH-sensitivity or other characteristics of liposomes and lipid particles.

4. Designing diagnostic and screening assays for protein misfolding diseases. For example, assay for mineral particles or for the relevant or disease-specific or disease-promoting lipid particles in samples of interest, such as blood, bodily fluids, or tissue.

Another example would be to test a given tissue or fluid sample for the capacity of some of the lipid particles to behave in a manner consistent with mineral-catalyzed lipid particles, e.g. to grow, change shapes, merge and/or divide, or for the presence of lipid particles, especially non-lamellar curved shape particles.

Another example would be the use of Mte or minerals to bind prions or misfolded disease proteins that are present in a biological sample; and thereby detect them.

5. Delivery of proteins or other cargo (for example, ribonucleic acid (RNA), deoxyribonucleic acid (DNA), peptides or other molecules) to cells using mineral-catalyzed lipid vesicles/liposomes, or using minerals to alter lipids, for example to promote uptake, or using lipids that form or promote the formation of non-lamellar curved shape lipid particles, again for example to promote uptake.

Alzheimer's Disease

In Alzheimer's disease, amyloid beta (A-beta) accumulates and is associated with neurodegeneration. Abeta results from proteolytic cleavage of amyloid precursor protein (APP). In the amyloidogenic pathway, APP in lipid rafts is cleaved first by Beta-secretase and then by gamma-secretase to generate Abeta. ABeta protein, and Beta and gamma-secretases are found in lipid rafts, along with GM1. In a the non-amyloidogenic pathway, APP is cleaved by alpha-secretase, producing alpha-CTF and soluble APP-alpha, which is released extracellularly. Alzheimer's disease is ultimately marked by Beta-amyloid (A-Beta) plaques, along with intracellular neurofibrillary tangles. Amorphous non-congo red positive plaques develop first, and later mature to B-sheet plaques. A-Beta fibrils aggregate into cross-beta-pleated-sheets. Abeta has been found in exosomes.

Gamma-secretase cleavage of APP may occur in acidic environments and the endosomal/lysosomal system, an important correlate with acidic and lipid vesicle environmental factors described herein. Abeta oligomers are considered to be critical to the pathogenesis of AD, and have been shown to be recruited to lipid rafts. ABeta peptide has a high affinity for copper, and Abeta to Abeta- peptide aggregation is stabilized by copper. Copper binding to Abeta is highly pH dependent. ApoE4 is correlated with increased AD risk. ApoE4 has been associated with lower metallothionein levels, possibly correlating increased copper or heavy metals with AD. ApoE4 may also affect lipid or vesicle disruption, distribution, or dispersion. Cholesterol levels are correlated with increased AD risk and appear to assist GM1 enrichment. Lipid oxidation products are reported to increase A-Beta aggregation. Contamination by trace amounts of metal such as copper also promotes A-beta aggregation.

Aluminosilicates have been found at the central core of AD plaques and also have been found in normal elderly brains. These were detected using energy dispersive X-Ray microanalysis (EDS) and solid-state nuclear magnetic resonance (MAS NMR). In AD, the form was amorphous, and the presence at the core of plaques rather than randomly distributed suggested that there may be relevance to pathology. Aluminosilicates have been capable of inducing Aβ aggregation and plaque formation in-vitro. Exposure to aluminosilicates in everyday life is common and thus uptake from environmental exposures is likely. Aluminosilicates may form in the body due to associations between particular minerals or mineral precursors. In addition to aluminosilicates, lipid rafts or liposomes may serve as a platform for AB aggregation.

Similarly to PrP-C and PrP-Sc in TSE, in AD, Abeta production relies on normal protein (APP) being cycled in from the plasma membrane and prion (Abeta) production is felt to occur largely in the trans-Golgi network (TGN), where, in AD, gamma-secretase may act. Interestingly, PrP-C regulates cleavage of APP, and this interaction may link the pathogenesis of the two diseases as well.

Negative Charges and Lipids

As with TSE disease, in AD, membranes are able to catalyze peptide (Abeta) changes, in particular for AD aggregation. The mechanism of interaction has been felt to involve binding of Abeta to GM1, dependent on the negatively-charged sialic acid residue, and causing a conformational shift from the alpha-helix structure to Beta-sheet structure as the peptide:lipid ratio increases. As with TSE, the negative charge is felt to be a large determinant of prion [Abeta] binding and pathology. GM1 is found tightly associated with ABeta, and this might represent an important cause of or seed for Abeta accumulation and aggregation. The helix-loop-helix structure also forms in SDS micelles.

Role of Copper in Misfolding

Abeta is an amphipathic peptide. Abeta self-aggregates easily, and accumulation/aggregation are enhanced by the presence of trace metals including copper, zinc, or iron, by oxidative damage of phosphatidylcholine (PC), in the presence of sphingomyelin (SM)-containing liposomes, and in the presence of negatively-charged phospholipids including phosphatidylserine (PS), phosphatidyl inisitol (PI), and cardiolipin. As with TSE disease where PrP-C binding to copper may facilitate transition to PrP-Sc, copper stabilizes Aβ aggregation and binds to the N-terminus of the peptide. Copper binding to Aβ increases subsequent oxidation of membrane components via enhancement of oxidative activity of the peptide as well as possible downstream generation of intermediates such as hydrogen peroxide, which contributes to AD end pathology. Copper has been found at a relatively high concentration in AD plaques. A-beta binds copper with high affinity, and demonstrates enzyme-like activity, oxidizing cholesterol and reducing O₂ to H₂O₂. Copper induces Abeta aggregation amorphously, and inhibits Beta-sheet structure formation. There is an acidic pH reported in AD plaques (˜5.4). Copper binds to Abeta and creates spherules while preventing B-sheets. Copper also binds to oxidized LDL, forming amyloid-protein staining positive structures.

The normal role of ApoE is to distribute lipids among cells. Apoe4, which has been associated with AD, has altered copper processing compared with the other apoE alleles. ApoE4 patients have lower metallothionein levels and less antioxidant activity than those with other ApoE variants. Metallothionein has a high binding affinity (scavenging) for copper, so reduced levels of metallothionein might increase copper levels. In AD, copper in the cells may react with glycine.

AD tissue does not show TVS, however, there are vacuolar structures. AD exhibits granulovacuolar degeneration (GVD) on electron microscopy, which shows an electron lucent vacuole in the cell containing an amorphous granule of unknown material.

Parkinson's Disease and Amyotrophic Lateral Sclerosis

Parkinson's disease again carries some striking overlaps with TSE and AD. PD is characterized by dopaminergic neuron loss. The neurons contain inclusions and protein aggregates of insoluble alpha-synuclein, a normally soluble protein. Like in TSE and AD, lipid interactions may lead to aggregation. Alpha-synuclein resides in lipid rafts and binds to GM1, promoting oligomer formation. As with AD and TSE, in PD, exosomal trafficking of abnormally folded protein is present. Oxidative stress is believed to play a role in both AD and PD and affects lipid raft stability. Alpha-synuclein also binds to copper. As with TSE, AD and PD, ALS also shows many of these features involving prion formation, derangement of lipids/lipid rafts, protein copper binding, and neurodegeneration.

It should be understood that the foregoing description is only illustrative of the invention. Various alternatives and modifications can be devised by those skilled in the art having the benefit of this disclosure, without departing from the invention. Accordingly, the invention is intended to embrace all such alternatives, modifications and variances.

Certain exemplary embodiments of the disclosure may be described. Of course, the embodiments may be modified in form and content, and are not exhaustive, i.e., additional aspects of the disclosure, as well as additional embodiments, will be understood and may be set forth in view of the description herein. Further, while the invention may be susceptible to various modifications and alternative forms, specific embodiments have been shown by way of examples and described in detail herein. However, it should be understood that the invention is not intended to be limited to the particular forms disclosed. Rather, the invention is to cover all modifications, equivalents and alternatives falling within the spirit and scope of the invention.

What is claimed is:

1. A method comprising preventing, diagnosing, or treating a protein misfolding disease by:
(a) detecting a mineral or a lipid particle, or
(b) removing or altering a binding or an interaction between any of the following: (i) a peptide causing protein-misfolding disease, (ii) a mineral, (iii) copper, and (iv) a lipid particle, or
(c) performing both step (a) and step (b).

2. The method of claim 1, wherein the peptide is PrP-C or PrP-Sc.

3. The method of claim 1, wherein the mineral is montmorillonite or aluminosilicate.

4. The method of claim 1, wherein the mineral is used as an aid in the detection or binding of disease proteins.

5. The method of claim 1, wherein a binding is released between (a) the peptide, (b) the mineral, (c) copper, or (d) a lipid particle.

6. The method of claim 1 further including the step of introducing a factor that binds competitively to the peptide, the mineral, the copper, or the lipid particle.

7. The method of claim 1, further including the step of inhibiting a copper binding condition.

8. The method of claim 1, including the step of using filtration or copper-scavenging agents to inhibit a copper binding condition.

9. A method comprising preventing, diagnosing, or treating a protein misfolding disease at a site including the step of:
(a) detecting lipid particles or the behavior of lipid particles, or
(b) removing or inhibiting lipid particles at the site by altering lipid particles, or their formation or growth, uptake, or spread; or
(c) performing both step (a) and step (b).

10. The method of claim 9, wherein the lipid particles are non-lamellar curved lipid particles or mineral-altered lipid particles.

11. The method of claim 9, further including the step of adding a modifier to reduce or clear lipid particles.

12. The method of claim 11, wherein the modifier is polyethylene glycol (PEG).

13. The method of claim 9, further including the step of adding a modifier to adjust a characteristic of liposomes or lipid particles.

14. The method of claim 13, wherein the characteristic is pH, lipid type, lipid environment, lipid shape, or circulation time.

15. The method of claim 9, further including the step of adjusting temperature to reduce, change, or inhibit lipid particles.

16. The method of claim 9, wherein lipid particles that form or promote the formation of non-lamellar curved-particle shapes are removed or inhibited.

17. The method of claim 9, wherein the formation of non-lamellar curved-particle shapes is inhibited.

18. The method of claim 9, wherein micelles or vesicles or non-lamellar curved-particle shapes or other lipid structures or shapes are disrupted.

19. A method of preventing or treating protein misfolding diseases at a site including the step of removing a mineral or a mineral-forming precursor from the site.

20. The method of claim 19 wherein the mineral is montmorillonite or aluminosilicate.

21. The method of claim 19 wherein the mineral or mineral-forming precursor is removed by filtration.

22. The method of claim 19 further including the step of assaying for a mineral or a mineral-forming precursor in blood, bodily fluids, or tissue for the purpose of prevention, diagnosis, or treatment.

23. A method comprising using a mineral, a mineral-catalyzed change, a lipid vesicle, or a lipid particle that can form a non-lamellar curved shape, to create misfolded proteins.

24. The method of claim 23, wherein the misfolded proteins are created in vitro.

25. The method of claim 23, wherein the misfolded proteins are created in vivo.

26. The method of claim 23, wherein the misfolded proteins are PrP-Sc, Abeta, alpha-synuclein, or SOD1.

27. The method of claim 23, wherein an animal model of a protein-misfolding disease is created.

28. The method of claim 27, wherein the protein-misfolding disease is one of the following: Transmissible spongiform encephalopathies, Alzheimer's disease, Parkinson's disease, and Amyotrophic lateral sclerosis.

29. A method including delivering proteins or other cargo to cells (a) using mineral-catalyzed lipid vesicles or liposomes, or (b) using minerals, or (c) using non-lamellar curved shape lipid particles or lipids that promote a non-lamellar curved shape, for example to promote uptake.

30. The method of claim 29, wherein the lipids are altered to promote uptake.
